(19) United States
(12) Patent Application Publication
(10) Pub. No.: US 2018/0344462 A1
(43) Pub. Date: Dec. 6, 2018

(54) Implantable Metallic Sheet for Bone Repair

(71) Applicant: Keun-Young Anthony Kim, Irvine, CA (US)

(72) Inventor: Keun-Young Anthony Kim, Irvine, CA (US)

(21) Appl. No.: 15/613,432

(22) Filed: Jun. 5, 2017

(51) Int. Cl. A61F 2/28; A61F 2/30; A61L 27/36; A61K 9/70

(52) U.S. Cl. CPC A61F 2/2846; A61F 2/30767; A61L 27/14; A61K 9/70; A61L 27/365

(57) ABSTRACT

A moldable sheet comprising malleable strands arranged in a substantially flat or planar configuration. The moldable sheet can be manipulated into a variety of shapes and is capable of maintaining the manipulated shape. Broken and fractured bones and bone fragments can be held together by wrapping a moldable sheet around the exterior of the break or fracture area. The moldable sheet can secure the ends of the bone for healing and can be incorporated into the new bone growth. The structure of the moldable sheet can be such that electromagnetic waves, such as those used with medical or security scanning equipment, are able to pass through pores in the device. This can make the moldable sheet radiolucent.

### BACKGROUND OF INVENTION

Broken bones and the associated severed blood vessels begin healing almost immediately after a break or fracture. During the healing process blood leaking from the vessels in the broken bone faun a clot of fibrous tissue around and between the area of the break. Chondrocytes begin to form collagen cells along strands of the fibrous tissue and eventually osteocytes move in and replace collagen with harder bone cells.

Some bone breaks require additional support to ensure that they heal properly. If the bone breaks in more than one area there may be disconnected fragments of bone, which need to be held in position, so that they can be incorporated into the bone during the regrowth process.

The usual method for joining and supporting the ends of a broken bone is to install brackets or plates secured with pins, screws, or other rigid mechanical devices across a break to align the bone and hold it in position. These devices are often left in place and become embedded in the regrown bone. The installation of such devices can create additional areas where bone needs to heal. For example, the use of screws or pins inserted into bone to secure a bracket or other device causes damage to the bone that has to heal in addition to the break or fracture area.

Such devices are typically not radiolucent and will appear on x-rays or other scans for the rest of the patient's life. Depending upon the size of the implanted devices, patients must be cognizant about advising anyone conducting a medical or security scan that they have an implant in that area of the bone. For this and other reasons, some patients develop a psychological discomfort with regard to the implants.

There is a need for an implantable device capable of joining and providing support to a broken bone that does not require a secondary attachment device, such as screws or pins, which are implanted into the bone tissue. It would be further advantageous if such device was radiolucent, so that it did not appear, or only minimally appears, on medical or security scans. Such a device could be easier to install and could alleviate patient discomfort and aversion to such devices.

### BRIEF SUMMARY

In accordance with embodiments of the invention, the problem of joining and supporting a broken bone in vivo, in a patient in need of such treatment, with an implant that does not require a secondary attachment mechanism to hold it to the bone tissue, is solved by the use of a woven or a random-mesh formed moldable sheet. The implantable moldable sheet can be ductile, malleable, pliable, formable, and/or bendable, so that it can be conformed to a shape and maintain the conformed or molded shape. The implantable moldable sheet can also be biocompatible and sterile. In this way, the implantable moldable sheet can be wrapped around, pressed against, or otherwise conformed to the shape of a bone or bone fragment and the material of the moldable sheet maintains the manipulated and formed position or shape without utilizing other secondary attachment mechanisms, such as screws, pins, bolts, or the like.

The implantable moldable sheet can also be porous, having multiple openings, to allow passage of in vivo materials, including, but not limited to, fluids, cells, drugs, nutrients, and other substances in the body. Advantageously, the porosity of the moldable sheet can also make it radiolucent, where it can permit at least partial passage or movement therethrough of electromagnetic radiation, including, but not limited to, light or radio waves, x-rays or other radiation, magnetic fields, proton or electron streams, or any of a variety of other signals, waveforms, or oscillations utilized by medical or security scans. Thus, while the strands of an implantable moldable sheet can be radiopaque, such that electromagnetic radiation is inhibited from passing there through, the pores permit sufficient passage of electromagnetic radiation to impart at least some radiolucency to the implantable moldable sheet.

In general, an implantable moldable sheet of the subject invention is a thin layer, two millimeters or less in thickness, of a plurality of strands of one or more materials. A strand can be elongated, such that the length is many times longer than the diameter. The material of a strand can be metallic, semi-metallic, polymer, plastic, or other natural or man-made materials. In one embodiment, at least one of the materials of a moldable sheet is capable of transmitting an electric current. In another embodiment, the moldable sheet is woven from strands or threads that cross over and under each other in a discernable pattern. For example, metallic strands can be formed into a woven pattern resembling that obtained by weaving techniques using a weft and warp, similar to that of fabric textiles.

In an alternative embodiment, the moldable sheet is one or more non-woven strands that are randomly arranged in a mesh or web of entangled, enmeshed, intertwined or otherwise irregular configuration.

The thickness, porosity, strength, and pliability or stiffness of an implantable moldable sheet can depend upon a variety of factors. For example, the thickness or diameter of the strands, the density of the strands, the number of strands, and other factors can all affect the characteristics of a moldable sheet. Further, the type of material utilized can affect the usable thickness of the strands. For example, titanium, gold, graphene, graphite, and alloys or combinations thereof can be used to form one or more of the strands of a moldable sheet and the physical characteristics of each of these materials can dictate the usable thickness and pliability of the strands. Preferably, the ductility and malleability of the materials or combinations thereof provide strands in a moldable sheet that can be formed tightly and compactly around a bone with minimal or no breakage of the strands.

The embodiments of the subject invention successfully address the disadvantages associated with the previously known implants for joining the ends of a broken bone within the body and providing support to the bone during healing and provide certain attributes and advantages, which have not been realized by those known devices. In particular, embodiments of an implantable moldable sheet of the subject invention is quicker and easier to use and can be retained in position on or around a bone, without the use of secondary attachment mechanisms that can further damage the bone or at least create additional areas of healing. The implantable moldable sheets can also be permeable or semi-permeable to medical or security scans, thereby making them invisible or partially-invisible, which can alleviate some patients' trepidations or aversions to the use of implants.

### BRIEF DESCRIPTION OF DRAWINGS

In order that a more precise understanding of the above recited invention can be obtained, a more particular description of the invention briefly described above will be rendered by reference to specific embodiments thereof that are illustrated in the appended drawings. The drawings presented herein may not be drawn to scale and any reference to dimensions in the drawings or the following description is specific to the embodiments disclosed. Any variations of these dimensions that will allow the subject invention to function for its intended purpose are considered to be within the scope of the subject invention.

FIG. 1 is an illustration of an embodiment of an implantable moldable sheet, according to the subject invention, formed from one or more randomly entangled or randomly oriented strands of a single material.

FIG. 2 is an illustration of an embodiment of an implantable moldable sheet, according to the subject invention, formed from two or more (solid lines and dashed lines) randomly entangled or randomly oriented strands of two different materials.

FIG. 3 is an illustration of an embodiment of an implantable moldable sheet, according to the subject invention, fainted from shorter sections of randomly entangled or randomly oriented strands, each of a different type of material.

FIG. 4 is an illustration of an embodiment of an implantable moldable sheet, according to the subject invention, formed from one or more woven strands of a single material.

FIG. 5 is an illustration of an embodiment of an implantable moldable sheet, according to the subject invention, formed from two or more woven strands (solid lines and dashed lines) of two different materials.

FIG. 6 is an illustration of an embodiment of an implantable moldable implant, according to the subject invention, formed from three woven strands (solid lines, dashed lines, heavy solid lines), each of a different type of material and with strands of each type of material having a different diameter.

FIG. 7 is a cross-sectional illustration of an embodiment of a moldable sheet of randomly entangled or randomly oriented strands, according to the subject invention, shown wrapped around a broken bone and closely conformed to the external shape of the bone ends.

FIG. 8 is a cross-sectional illustration of an embodiment of a moldable sheet of woven strands, according to the subject invention, shown wrapped around a broken bone and closely conformed to the external shape of the bone ends.

FIG. 9 is a cut-away view of a bone that has been wrapped with a moldable sheet of entangled or randomly oriented strands, where the moldable sheet has been incorporated into the new bone tissue. Also shown is the passage through the moldable sheet and bone of the electromagnetic waves generated by a medical or security scanner.

FIG. 10 is a side cut-away view of a bone that has been wrapped with a moldable sheet of woven strands, where the moldable sheet has been incorporated into the new bone tissue. Also shown by the arrows is the passage through the moldable sheet and bone of the electromagnetic waves generated by a medical or security scanner.

FIGS. 11A and 11B are side elevation views of embodiments of a moldable sheet that have areas that are thicker than other areas of the moldable sheet. FIG. 11A shows a moldable sheet that is thicker at one end than in another. As shown here, the lower end of the sheet is thicker than the upper end. FIG. 11B shows a moldable sheet that is thicker in the middle than at the upper and lower ends.

FIG. 12 shows an example of a bone spacer with an implantable moldable sheet of the subject invention shaped to fit inside a lumen of the spinal spacer.

FIG. 13 illustrates an embodiment of an implantable moldable sheet wrapped around a bone spacer and the bone ends between which it is inserted.

FIG. 14 illustrates another embodiment of an implantable moldable sheet in the form of a long strand or tape that is wound around bone.

### DETAILED DISCLOSURE

The subject invention pertains to implants for securing the ends or fragments of a broken bone to promote proper healing. More specifically, the subject invention provides one or more embodiments of an implantable moldable sheet, or similar device, which are wrapped around a bone to secure broken ends or fragment of the bone in place during healing. The implant can remain in place on the bone and eventually be incorporated into the new bone growth. The structure of the moldable implant can be such that it has minimal or no visibility on medical or security scanner devices. The subject invention is particularly useful in the field of orthopedic procedures, in particular devices used for the joining and support of broken or fractured bones.

In the description that follows, a number of terms are used. In order to provide a clear and consistent understanding of the specification and claims, including the scope to be given such terms, the following definitions are provided.

The term “patient” as used herein, describes an animal, including mammals, to which the devices and methods of the present invention can be applied. This includes mammalian species such as, but not limited to, apes, chimpanzees, orangutans, humans, and monkeys; domesticated animals (e.g., pets) such as dogs, cats, guinea pigs, and hamsters; large animals such as cattle, horses, goats, and sheep; and, any wild or non-domesticated animal.

The present invention is more particularly described in the following examples that are intended to be illustrative only because numerous modifications and variations therein will be apparent to those skilled in the art. As used in the specification and in the claims, the singular for “a,” “an” and “the” include plural referents unless the context clearly dictates otherwise.

Reference will be made to the attached figures on which the same reference numerals are used throughout to indicate the same or similar components. With reference to the attached figures, which show certain embodiments of the subject invention, it can be seen that the subject invention can comprise a flat, planar sheet 10 formed of one or more strands 20 where at least one of the strands has ductility and is pliable, malleable, moldable, shapeable, or otherwise conformable and can be manipulated into a shape or form with minimal or no breaking and can maintain that shape or form indefinitely. In one embodiment, the sheet is sterile. In a further embodiment, the sheet comprises one or more biocompatible materials.

In one embodiment, a moldable sheet has one or more strands of at least one material with electro-conductive properties. In a particular embodiment, a moldable sheet has electro-conductive strands 30 of one or more formable or moldable materials that can transmit an electric current either directly, such as, for example, by direct attachment to an electrical current source, or indirectly, such as, for example, by stimulation from an electromagnetic energy source.

The moldable sheets 10 can further comprise biocompatible materials. Such biocompatible materials can be further sterile or sterilizable. In one embodiment, a moldable sheet is formed with strands of one or more biocompatible metallic materials, such as, for example, titanium, titanium alloys, tungsten, tungsten alloys, stainless steel, gold, and combinations thereof. In another embodiment, a moldable sheet is formed with strands of one or more semi-metallic materials, such as, for example, graphite, graphene, or alloys, composites, or mixtures of these or other materials. In yet another embodiment, a moldable sheet is formed with strands of polyether ether ketone (PEEK) polymer. Other biocompatible materials having the malleability and ductility necessary for forming or molding a sheet around a bone can also be used. A person with skill in the art can determine one or more materials suitable for the strands of a moldable sheet. Such variations are within the scope of the subject invention.

A moldable sheet of the subject invention is particularly advantageous for wrapping around and securing the ends and any fragments, if present, of a broken or fractured bone. During the healing process, the moldable sheet can become incorporated into the new bone growth. For example, all or part of the moldable sheet can become covered or encased in the new bone growth. As such, it can be important that the moldable sheet occupy as little space as possible around the bone, so that the resulting healed area does not form a bulge or bone spur.

The strands of a moldable sheet can have the smallest possible diameter achievable for a given material that still provides sufficient ductility and malleability to maintain a formed or molded shape, as described above. The smallest achievable diameter can vary between materials. For example, titanium alloy strands can have a usable diameter as small as 0.0254 mm and gold strands can have a usable diameter as small as 0.018 mm. PEEK strands can vary in how small a usable diameter they can have based on the strand processing method. In one embodiment, any strand in a moldable sheet can be between approximately 0.001 mm and approximately 1.0 mm. In a more specific embodiment, any strand in a moldable sheet is at least 0.001, at least 0.005, at least 0.01, at least 0.05, at least 0.1, at least 0.5, and at least 1 mm in diameter and/or a diameter in a range between any two of the listed values.

In one embodiment, the strands of a moldable sheet are radiopaque, such that they inhibit or prevent the passage of electromagnetic radiation therethrough. In a further embodiment, the diameter of the strands can be such that the wavelength of the electromagnetic radiation bypasses, goes around, or is at least not inhibited by the strands. As discussed herein, the pores between the strands can have a diameter that permits passage of electromagnetic radiation.

Cells, particularly the cells involved with bone growth (e.g., chondrocytes and osteocytes), have an affinity for rough, imperfect or unsmooth surfaces, on which it can be easier for such cells to form attachments. In one embodiment, one or more of the strands have unsmooth or unpolished surfaces. In an alternative embodiment, one or more of the strands is manufactured to have specific surface features or imperfections, such as, for example, indentations, irregularities, roughened surface textures, or other surface treatments that can facilitate cell attachment.

The strands 20 of a moldable sheet 10 can be combined or interwoven or intertwined into any of a variety of configurations. In one embodiment, the strands are non-woven or are randomly arranged so that they overlap and/or intertwine with each other and do not form a discernable pattern, such as shown in FIGS. 1-3. For example, certain spun-form or extrusion techniques can layer and intertwine a plurality of strands in a random arrangement. In another embodiment, the plurality of strands is woven using a weft and warp, such as shown, for example in FIGS. 4-6, such that the strands foil a discernable pattern. The moldable sheet can have a homogeneous composition, such that all of the strands comprise the same material. Alternatively, a moldable sheet can have a heterogeneous composition, such that strands comprise different materials.

The ratios of strands of different materials can also vary depending upon a variety of factors understood by those with skill in the art. For example, one material may have certain advantageous and disadvantageous characteristics and another material may also have certain other different advantageous and disadvantageous characteristics. When combined, the advantageous characteristics of one material can offset or compensate for the disadvantageous characteristics of the other material.

It is also possible for different areas of a moldable sheet to have strands of different materials in different ratios. For example, it can be advantageous for certain areas of a moldable sheet to have characteristics that are different from other areas of a moldable sheet. By way of further example, it can be advantageous for an area near one or more edges to be more pliable or have more ductility than an area near one or more other edges of a moldable sheet. In a further example, it may be advantageous for certain areas of a moldable sheet to have more electro-conductive fibers than another area of a moldable sheet. Thus, some areas may have more titanium or steel strands or strands of different diameter than those of another area of the moldable sheet.

In one embodiment, the arrangement of strands of different materials is uniform throughout the moldable sheet. In another embodiment, the arrangement of strands of is non-uniform, such that there are areas with strands of different materials in different ratios. This can provide a moldable sheet with areas having different material characteristics or properties than other areas. FIG. 3 illustrates a non-limiting example of a molded sheet having strands of different materials and arranged in different ratios in a moldable sheet. In this illustration the middle section is less dense or has fewer strands than the left and right edges.

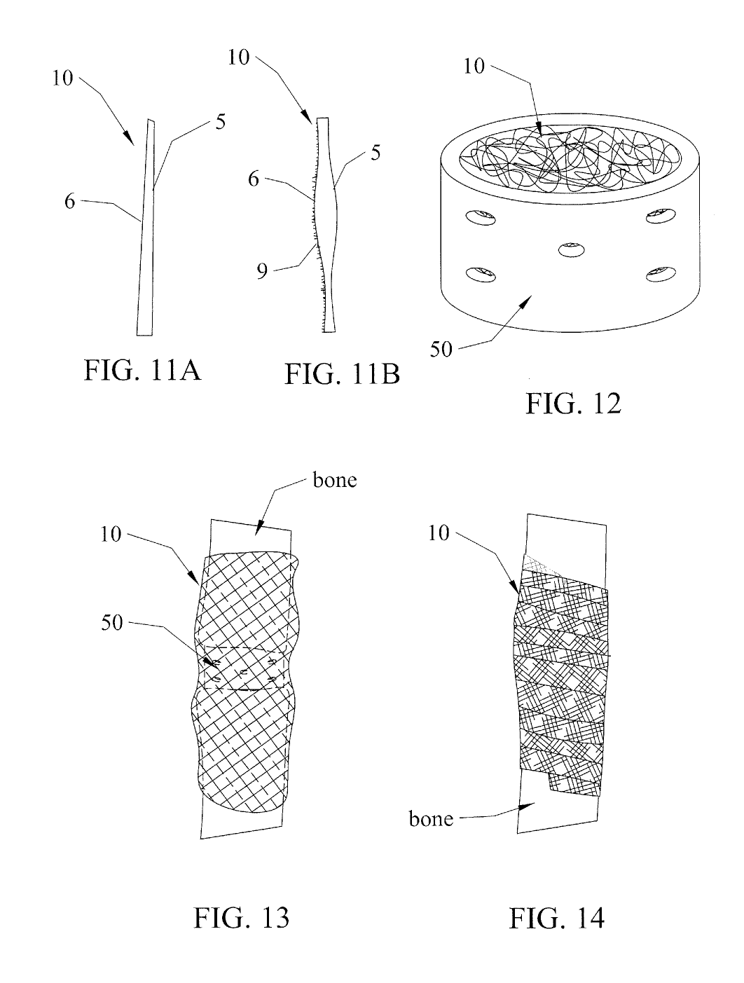

It can also be advantageous for certain areas of a moldable sheet to be thicker than other areas of a moldable sheet, wherein thickness is the distance between the top surface 5 and the bottom surface 6 of a moldable sheet 10. FIG. 11 A shows a non-limiting example of a sheet that is thicker towards one end. FIG. 11B shows a non-limiting example of a sheet that is thicker along the sides than in the middle. Thickness can be imparted by increased strand diameter. Thickness can also be imparted by having more strands in a particular area.

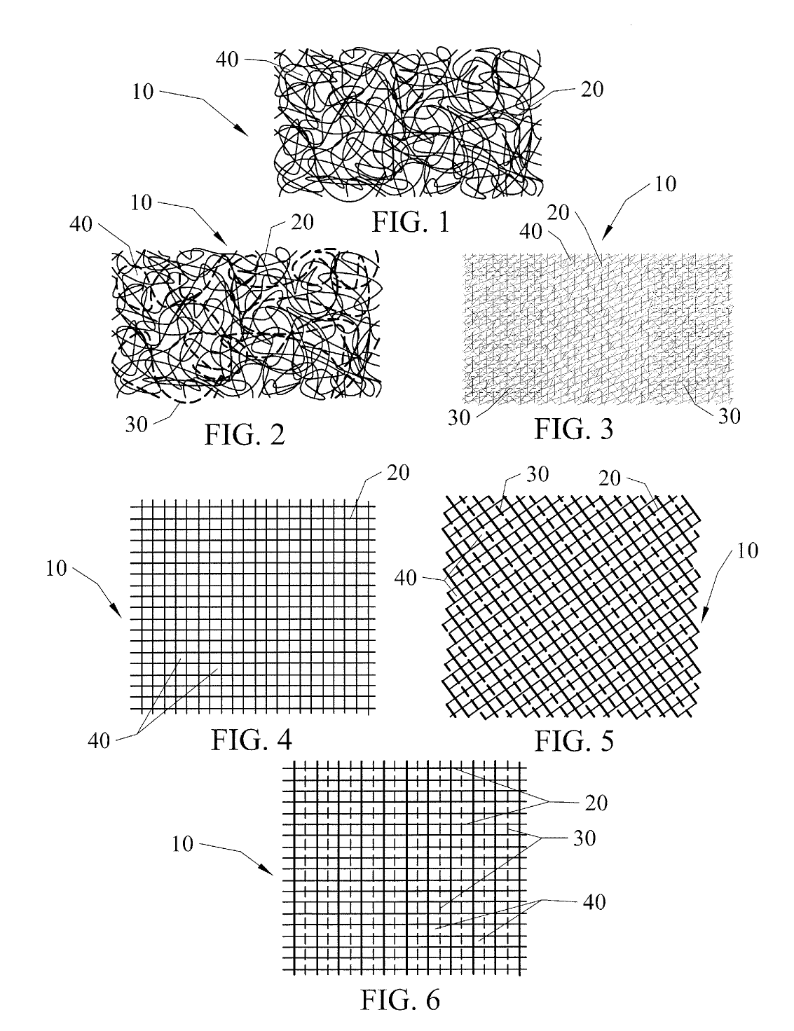

In one embodiment, a moldable sheet is formed by the random arrangement of non-woven strands combined in at least one of an overlapping, interlacing, and intertwining configuration, where there is no discernable pattern to the arrangement of the strands. FIGS. 1, 2, and 3 illustrate examples of non-woven moldable sheets. In a further embodiment, the strands are elongated, continuous or substantially continuous, so that they make multiple turns and/or overlappings, as illustrated, by way of example, in FIGS. 1-2.

In an alternative embodiment, the plurality of strands of a moldable sheet are short pieces or sections that are randomly overlapping, interlacing, intertwining or otherwise interconnected, which is shown, for example, in FIG. 3. In one embodiment, the top surface 5 of a moldable sheet 10, which would be directed away from a bone surface, to be smooth and have minimal or no protruding strands that can penetrate surrounding tissue. In a further embodiment, the bottom surface 6 is rough or has protruding strand ends 9, which is illustrated, for example, in FIG. 11B. With this embodiment, a moldable sheet can be wrapped so that the protruding strand ends 9 face towards a bone, so that they overlap with the moldable sheet to further facilitate the moldable sheet maintaining or holding position on and around a bone. Preferably the strands of a moldable sheet are sufficiently long and/or pliable that when the moldable sheet is manipulated into a particular shape, the ends of the strands curve with the sheet, so as to have minimal or no protruding ends of strands that can create a bristled or spiny surface.

In another embodiment, a moldable sheet is woven of strands forming a pattern resembling that obtained by weaving techniques using a weft and warp, similar to that of cloth or fabric textiles. FIGS. 4, 5 and 6 illustrate non-limiting examples of woven moldable sheets. In one embodiment, a moldable sheet is woven from strands of all the same material, such as shown in FIG. 4. Alternatively, there can be strands of two or more different materials, where, for example, the warp comprises strands of one material or strands in a combination of two or more materials and the weft comprises strands of one material or strands in combination of two or more materials. FIG. 6 illustrates an example where the warp has strands of one material and the weft has strands of two different materials. FIG. 5 illustrates an example of an embodiment where the strands are cross-woven and where the warp has strands of two different materials and the weft comprises strands of one material. Other combinations of strands can also be used. Such variations are within the scope of the subject invention.

Advantageously, a moldable sheet of the subject invention can be radiolucent, such that it allows passage of electromagnetic radiation. This radiolucency means that a moldable sheet can present a minimal or no image on medical or security scanner devices. The level of radiolucency of a moldable sheet can be dictated by the porosity of the moldable sheet, that is, the number and area encompassed by the spaces, openings or pores 40 between the strands 20 of the moldable sheet 10 that allow passage of electromagnetic radiation. Thus, the radiolucency of a moldable sheet can depend upon the characteristics of the strands, their arrangement in the moldable sheet, the density or number of the strands in a moldable sheet, and how closely the strands are to each other, all of which can dictate the level of porosity of the moldable sheet.

FIG. 9 is an illustration of a bone in which an implantable moldable sheet of random strands has been incorporated into bone as it healed and how electromagnetic waves 45 from a scanner can pass through the bone and moldable sheet. FIG. 10 is an illustration of a bone in which an implantable moldable sheet of woven strands has been incorporated into the bone as it healed and how electromagnetic waves from a scanner pass through the bone and the moldable sheet. The illustration in FIG. 10 also demonstrates an example of how less electromagnetic radiation can pass through an implantable moldable sheet of tightly woven strands than through looser woven random strands shown in FIG. 9. Alternatively, if the woven strands are looser than the random strands, more electromagnetic radiation can pass through the woven strands. But, with either moldable sheet, at least some electromagnetic radiation can pass through the pores 40.

In one embodiment, strands 20 of a moldable sheet are configured with a plurality of pores, wherein the pores 40 vary in shape. In a more specific embodiment, the strands of a moldable sheet are configured so that the shape of the pores is varied, but the area of the pores is approximately the same. For example, a moldable sheet of non-woven strands can have a plurality of pores of different shapes, but the same or similar areas. Alternatively, a moldable sheet of non-woven strands can have pores of different shapes and different areas. In one embodiment, the areas of pore sizes in a moldable sheet vary between approximately 5% and approximately 100%. In another embodiment, the areas of pore sizes in a moldable sheet vary between approximately 20% and approximately 80%. In yet another embodiment, the areas of pore sizes in a moldable sheet vary between approximately 40% and approximately 60%. In a specific embodiment, the areas of pore sizes in a moldable sheet vary approximately 50%.

In an alternative embodiment, strands 20 of a moldable sheet are configured so that pores 40 have similar shapes. In a more specific embodiment, the strands of a moldable sheet are configured so that the shape of the pores is the same or similar and the area of pores is also the same or similar. For example, a moldable sheet of woven strands can have pores that are rectangular in shape and can have the same or similar areas. In one embodiment, the area of the pores of a moldable sheet of woven strands varies between approximately 1% and approximately 25%. In another embodiment, the area of the pores of a moldable sheet of woven strands varies between approximately 5% and approximately 20%. In yet a further embodiment, the areas of the pores of a moldable sheet of woven strands varies between approximately 10% and 15%.

Electromagnetic bone growth stimulation can be used to enhance bone healing and improve outcomes with implants and procedures. Bone growth stimulation can use invasive, semi-invasive, or non-invasive devices to generate a current in an implant. Typically, metallic or semi-metallic implants are utilized with such devices to create an electrical current across a break or fracture to stimulate bone growth in the direction of the current. The embodiments of the subject invention are conducive for use with electromagnetic bone growth stimulation devices.

In one embodiment, a moldable sheet 10 comprises one or more electro-conductive strands 30 in which an electric current can be induced indirectly, such as, for example, with pulsed electromagnetic field (PEMF) generators. In an alternative embodiment, a moldable sheet comprises one or more electro-conductive strands in which an electric current can be directly generated, such as, for example, with capacitive coupling devices. There are other devices known and used to generate electric current for the purpose of stimulating bone growth. A person with skill in the art, having benefit of the subject disclosure, can determine the appropriate device for use with a particular patient and with an embodiment of a moldable sheet. Such variations are within the scope of the subject invention.

In some situations, a rigid implant can be used between bone ends, particularly where there is missing bone tissue. If the gap between bone ends exceeds 50% of the diameter of the bone ends, it can be beneficial to install an implant in the gap to encourage bone healing. These implants are often rigid bodies that are fixed to the bone in which bone tissue migrates from each end. The moldable sheet can be used with such devices to facilitate or enhance the healing process. For example, polyether ether ketone (PEEK) polymers are being increasingly used for implants. In addition to being biocompatible, PEEK has certain other benefits such as being light-weight, radiolucent, stronger than most metals, an elastic modulus similar to that of human bone, and being capable of precise machining. PEEK is also bio-inert making it difficult to integrate with adjacent bone tissue. PEEK is also not electro-conductive and cannot generate an electric current with electromagnetic stimulation devices. Embodiments of a moldable sheet of the subject invention can be utilized in conjunction with a PEEK implant to facilitate bone growth and compensate for the lack of integration by a PEEK implant. For example, spinal cages 50 are often used to replace or fuse together vertebrae. The moldable sheet 10 can be incorporated into the central lumen or other spaces in a spinal cage or other type of bone spacer. FIG. 12 illustrates a non-limiting example of a spinal cage in which a moldable sheet has been conformed, compressed, or otherwise molded and inserted into the central lumen. A moldable sheet can also be wrapped around the area of the PEEK implant. Advantageously, this can provide the benefits of both devices and materials, including maintaining the radiolucent properties of both devices. FIG. 13 illustrates a non-limiting example of a bone spacer between bone ends and wrapped with a specific embodiment of a moldable sheet.

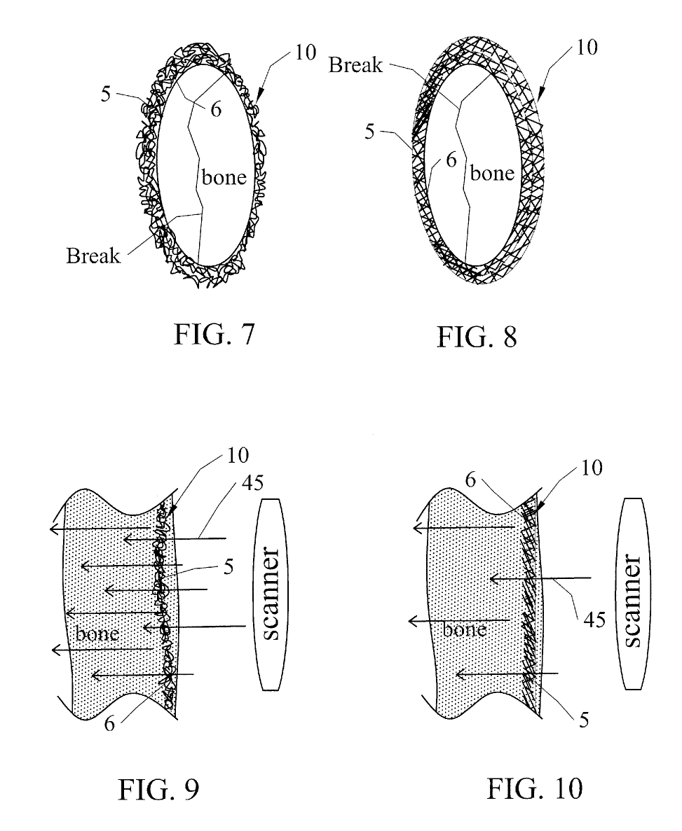

A moldable sheet of the subject invention can be most effective when in close proximity to the bone, such as shown, for example, in FIGS. 7 and 8. This can aid in aligning the bone ends and any fragments and inhibiting the formation of large bone spurs or defoimities in the bone. A moldable sheet can be wrapped one or more times around a bone, with each wrapping increasing the strength and rigidity. The strands can also become entangled between each layer, which also assists in maintaining the manipulated shape and form. Soft tissue around the break is usually dissected away to maximize contact between the moldable sheet and the bone.

Preferably, the moldable sheet has dimensions that allow it be wrapped around a bone sufficient times to maintain the bone position, with minimal excess material. In one embodiment, a moldable sheet is formed in one or more dimensions and shapes. In an alternative embodiment, a moldable sheet can be cut to a required size and shape from a larger sheet.

In one embodiment, a moldable sheet has a length of between approximately 1″ and approximately 12″ and a width of between approximately 1″ and approximately 12″. Thus, a moldable sheet can be, for example, a square or rectangular shape that can be wrapped band-like around a bone, such as shown, for example, in FIG. 13. In an alternative example, a moldable sheet can be an elongated, tape-like form that can be wound around a bone, such as shown, for example, in FIG. 14. It can be beneficial, though not required, that the ends and edges of a moldable sheet overlap when wrapped or wound around a bone, as demonstrated in FIG. 14.

Embodiments of a moldable sheet, according to the subject invention, comprise a substantially flat or planar device that can be manipulated into a variety of shapes and is capable of holding or maintaining the manipulated shape. A moldable sheet of the subject invention can be advantageous for wrapping and conforming around a tissue, particularly bone tissue. Broken and fractured bones and bone fragment can be held together by wrapping a moldable sheet around the exterior of the break or fracture area. The moldable sheet can secure the ends of the bone for healing and can be incorporated into the new bone growth. The structure of the moldable sheet can be such that electromagnetic waves, such as those used with medical or security scanning equipment, are able to pass through pores in the device. This can make the moldable sheet more radiolucent, and less visible with such scanning devices.

All patents, patent applications, provisional applications, and other publications referred to or cited herein are incorporated by reference in their entirety, including all figures and tables, to the extent they are not inconsistent with the explicit teachings of this specification. Additionally, the entire contents of the references cited within the references cited herein are also entirely incorporated by reference.

Any reference in this specification to “one embodiment,” “an embodiment,” “example embodiment,” “further embodiment,” “alternative embodiment,” etc., is for literary convenience. The implication is that any particular feature, structure, or characteristic described in connection with such an embodiment is included in at least one embodiment of the invention. The appearance of such phrases in various places in the specification does not necessarily refer to the same embodiment. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

1. A sterile implantable moldable sheet, adapted for wrapping and securing a bone, comprising:
a plurality of interwoven strands of at least one malleable, shape-retaining, electro- conductive material, such that the sheet will maintain a formed shape when wrapped around a bone; and
a plurality of pores of sufficient size between the strands to permit passage of electromagnetic radiation,
such that, when the sheet is wrapped around the bone, sufficient electromagnetic radiation passes through the plurality of pores that the sheet is at least partially radiolucent.

2. The sterile implantable moldable sheet, according to claim 1, wherein the one or more strands are randomly intertwined without forming a discernable pattern to the strands.

3. The sterile implantable moldable sheet, according to claim 1, wherein the one or more strands are intertwined by being woven, so that the strands form a discernable pattern.

4. The sterile implantable moldable sheet, according to claim 2, where all of the strands comprise the same material.

5. The sterile implantable moldable sheet, according to claim 2, where one or more of the strands comprise a different material from one or more other strands.

6. The implantable moldable sheet, according to claim 4, wherein the thickness is between 0.1 mm and 2.0 mm.

7. The sterile implantable moldable sheet, according to claim 5, wherein at least one of the strands is a different electro-conductive material than the electro-conductive material of another strand.

8. The sterile implantable moldable sheet, according to claim 1, wherein the strands are radiopaque.

9. The sterile implantable moldable sheet, according to claim 5, wherein the strands are unpolished.

10. A method for treating a broken bone utilizing an implantable moldable sheet comprising:
positioning an implantable moldable sheet, according to claim 1, in area of the broken bone;
forming the implantable moldable sheet to the area of the broken bone, so that the implantable moldable sheet is incorporated into the bone tissue formed during healing of the broken bone.

11. The method according to claim 10, further comprising dissecting soft tissue from the area of the broken bone to facilitate forming the implantable moldable sheet in contact with the area of the broken bone.

12. The method according to claim 10, further comprising operably attaching an electromagnetic bone growth stimulator to the implantable moldable sheet to create an electric current in the at least one electro-conductive strand in the implantable moldable sheet.
